(19) United States
(12) Patent Application Publication
(10) Pub. No.: US 2024/0021276 A1
(43) Pub. Date: Jan. 18, 2024

(54) DATA PREPROCESSING SYSTEM FOR CLEANING SMALL MOLECULE COMPOUND AND METHOD THEREOF

(71) Applicant: Ainnocence INC, MIAMI, FL (US)

(72) Inventors: Yang Jiao, Shanghai (CN); Lurong Pan, Vestavia Hill, AL (US)

(21) Appl. No.: 18/315,516

(22) Filed: May 11, 2023

(30) Foreign Application Priority Data
Jul. 18, 2022 (CN) 2022108440536

(51) Int. Cl. G16C 20/30; G16C 20/50

(52) U.S. Cl. CPC G16C 20/30; G16C 20/50

(57) ABSTRACT

The present invention provides a data preprocessing method for cleaning a small molecule compound, the data preprocessing method comprising: an S1 text preprocessing step including: preprocessing an original SMILES text of a small molecule compound into a standardized SMILES text of the small molecule compound; and an S2 chemical graph formatting step including: splitting in a format each text element of the standardized SMILES text of the small molecule compound of S1 to obtain chemical graph information of the small molecule compound. The present invention also provides a data preprocessing system for cleaning a small molecule compound. The present invention enables the cleaning, deduplication, and standardization of global datasets, providing an efficient, fast, accurate integration method for the cleaning of end-to-end small molecule compounds.

### CROSS REFERENCE TO RELATED APPLICATIONS

This Application claims priority from a patent application filed in China having Patent Application No. CN2022108440536 filed on 18 Jul. 2022 and titled “DATA PREPROCESSING SYSTEM FOR CLEANING SMALL MOLECULE COMPOUND AND METHOD THEREOF”.

### TECHNICAL FIELD OF THE INVENTION

The present invention belong to the field of medicine and artificial intelligence, and more particularly, to a data preprocessing system for cleaning a small molecule compound and a method thereof.

### BACKGROUND OF THE INVENTION

In traditional methods, the compounds are standardized based on chemical informatics methods to obtain cleaning methods for a small molecule compound. However, with the arrival of the era of large data, the requirements of high efficiency, accuracy and fast computing speed are put forward. The traditional algorithms based on the chemical informatics are inefficient and cannot meet the needs of large data age, and the data standards of various open source algorithms are not unified.

In particular, there are now numerous sources of SMILES compound information (e.g., open source databases such as Chembl, pubChem, etc.), which lack unified and standardized operations. It cannot well distinguish clean and unclean data for duplication. In addition, there are currently methods of a partial cleaning and a de-duplication process based on the rules. The process is directed to building databases only, whiteout practical application downstream (e. g., machine learning or deep learning). Non-standard or repetitive structures can still be encountered with this method.

Furthermore, the mathematical graphs for now translating SMILES to graph neural networks lack standardization, and the algorithms called from individual open source frameworks lack uniform standards. Based on the above, the present application provides a technical solution for solving the above technical problem.

### SUMMARY OF THE INVENTION

It is a first object of the present invention to provide an efficient, fast, accurate integrated method for the cleaning of end-to-end small molecule compounds. A second object of the present invention is to achieve an efficient, fast, accurate integrated system for the cleaning of end-to-end small molecule compounds. A first aspect of the invention provides a data preprocessing method for cleaning a small molecule compound, the data preprocessing method comprising: an S1 text preprocessing step including: preprocessing an original SMILES text of a small molecule compound into a standardized SMILES text of the small molecule compound according to predetermined text processing rules; and an S2 chemical graph formatting step including: splitting in a format each text element of the standardized SMILES text of the small molecule compound of S1 according to the predetermined text processing rules to obtain a digitized graph structure of chemical information of the small molecule compound.

In a preferred embodiment of the present invention, the method further comprises a step S3, wherein the digitized graph structure of the chemical information of the small molecule compound of S2 is used for the construction of an artificial intelligence model.

In a preferred embodiment of the invention, when the original SMILES text of the small molecule compound is preprocessed into the standardized SMILES text of the small molecule compound in the S1 text preprocessing step, the predetermined text processing rules comprises: step S1-1, optional structural normalization, wherein the data of the small molecule compound is processed into the original SMILES text; step S1-2, if the original SMILES text comprises heavy metal components and organic compound components, removing the heavy metal components from and retaining the organic compound components in the original SMILES text; step S1-3, if the original SMILES text comprises multimer components, removing the multimer components from and retaining a longest component in the original SMILES text; step S1-4, if the original SMILES text comprises a charge, adding or subtracting a hydrogen atom in the original SMILES text to remove the charge; step S1-5, removing special SMILES text information; and step S1-6, exporting normalized sequences to obtain the normalized SMILES text for the small molecule compound.

In a preferred embodiment of the present invention, when each text element of the standardized SMILES text of the small molecule compound of S1 is split in a format in the S2 chemical graph formatting step, the predetermined text processing rules comprise: step S2-1, splitting the standardized SMILES text of the small molecule compound of S1 into text elements of each core to obtain text elements of the small molecule compound; step S2-2, performing text processing and identification on the properties of the text elements of the small molecule compound of step S2-1, and identifying and completing simplified chemical information to obtain a chemical information graph of the small molecule compound; step S2-3, according to the chemical information graph of the small molecule compound in step S2-2, establishing a coordinate system with an atomic element as a node, and constructing a digital coordinate system of the chemical information graph of the small molecule compound; step S2-4, according to the digital coordinate system of the chemical information graph of the small molecule compound in step S2-3, adding element attributes of nodes and edges to obtain a digitized graph structure of the chemical information of the small molecule compound.

In a preferred embodiment of the present invention, step S2-5 includes complementing the hydrogen atom information of the digitized graph structure of the chemical information, if necessary.

In a specific embodiment, it further comprises step S2-6, completely exporting a digitized graph structure of chemical information of the small molecule compound.

A second aspect of the invention provides data preprocessing system for cleaning a small molecule compound adapted for the data preprocessing method according to any one of claims 1 to 5, comprising: an S1 text preprocessing unit configured to include preprocessing original SMILES data of the small molecule compound into a standardized SMILES text of the small molecule compound according to predetermined text processing rules; and an S2 chemical graph formatting unit configured to include: splitting in a format each text element of the standardized SMILES text of the small molecule compound of S1 according to the predetermined text processing rules to obtain a digitized graph structure of chemical information of the small molecule compound.

In a preferred embodiment of the present invention, it further comprises an S3 unit configured such that a digitized graph structure of the chemical information of the small molecule compound of S2 is used in the construction of an artificial intelligence model.

In a preferred embodiment of the present invention, when the original SMILES text of the small molecule compound is preprocessed into the standardized SMILES text of the small molecule compound in the S1 text preprocessing unit, the predetermined text processing rule comprises: an S1-1 unit configured for optional structural normalization, wherein the data of the small molecule compound is processed into the original SMILES text; an S1-2 unit configured for, if the original SMILES text comprises heavy metal components and organic compound components, removing the heavy metal components from and retaining the organic compound components in the original SMILES text; an S1-3 unit configured for, if the original SMILES text comprises multimer components, removing the multimer components from and retaining a longest component in the original SMILES text;

an S1-4 unit configured for, if the original SMILES text comprises a charge, adding or subtracting a hydrogen atom in the original SMILES text to remove the charge; an S1-5 unit configured for removing special SMILES text information; and an S1-6 unit configured for exporting normalized sequences to obtain the normalized SMILES text for the small molecule compound.

In a preferred embodiment of the present invention, when each text element of the standardized SMILES text of the small molecule compound of S1 is split in a format in the S2 chemical graph formatting unit, the predetermined text processing rules comprise: an S2-1 unit configured for splitting the standardized SMILES text of the small molecule compound of S1 into text elements of each core to obtain text elements of the small molecule compound; an S2-2 unit configured for performing text processing and identification on the properties of the text elements of the small molecule compound of the S2-1 unit, and identifying and completing simplified chemical information to obtain a chemical information graph of the small molecule compound; an S2-3 unit configured for, according to the chemical information graph of the small molecule compound in the S2-2 unit, establishing a coordinate system with an atomic element as a node, and constructing a digital coordinate system of the chemical information graph of the small molecule compound; an S2-4 unit configured for, according to the digital coordinate system of the chemical information graph of the small molecule compound in the S2-3 unit, adding element attributes of nodes and edges to obtain a digitized graph structure of the chemical information of the small molecule compound.

In a preferred embodiment of the invention, an S2-5 unit configured for, if necessary, complementing the hydrogen atom information of the digitized graph structure of the chemical information, if necessary. In a specific embodiment, it further comprises a unit S2-6, completely exporting a digitized graph structure of chemical information of the small molecule compound. A third aspect of the invention provides an electronic device comprising a memory and a processor, wherein the memory is configured to store one or more computer instructions which, when executed by the processor, implement the data preprocessing method for cleaning a small molecule compound of the present invention.

The present invention can bring at least one of the following benefits. The method of the present invention is based on the combination of large data and natural language processing technology with a part of chemical informatics to achieve a new method that can achieve lower computational costs, and ultimately achieve more accurate data preprocessing and more convenient downstream use.

### BRIEF DESCRIPTION OF DRAWINGS

The above characteristics, technical features, advantages and the manner of implementing them will be more clearly understood from the following description of preferred embodiments taken in conjunction with the accompanying drawings.

FIG. 1 is a flow chart for a data processing method of the present invention (with two separate but associable parts);

FIG. 2 is a flow chart for the operation of the present invention; and

FIG. 3 is a schematic diagram of data variable conversion in the present invention.

### DETAILED DESCRIPTION

Various aspects of the invention are described in further detail below.

Unless otherwise defined or indicated, all technical and scientific terms used herein have the same meaning as commonly understood by one of ordinary skill in the art to which this invention belongs. In addition, any methods and materials similar or equivalent to those described can be used in the methods of the present invention.

Terms are described below. As used herein, the term “or” includes the relationship of “and” unless specifically stated or limited otherwise. The “and” corresponds to a Boolean logic operator “AND”, the “or” corresponds to a Boolean logic operator “OR”, and “AND” is a subset of “OR”.

It will be understood that, although the terms first, second, etc. may be used herein to describe various elements, these elements should not be limited by these terms. These terms are only used to distinguish one element from another. Thus, a first element may be termed a second element without departing from the teachings of the inventive concept.

As used herein, the terms “containing”, “comprising”, or “including” mean that the various ingredients may be used together in a mixture or composition of the present invention. Thus, the terms “consisting essentially of” and “consisting of” are encompassed by the terms “containing”, “comprising”, or “including”.

Unless specifically stated or limited otherwise, the terms “connected with”, “communicated”, and “connecting” are to be construed broadly, e.g., as a fixed connection, as a connection through an intervening medium, as a connection between two elements, or as an interaction between two elements. The specific meaning of the above terms in this application will be understood in specific circumstances by those of ordinary skill in the art.

For example, if an element is referred to as being on, coupled to, or connected to another element, it can be directly formed on, coupled to, or connected to the other element; or intervening elements may be present therebetween. In contrast, if the phrases “directly on”, “directly coupled to”, and “directly connected to” are used herein, there are no intervening elements present. Other words used to describe the relationship between elements should be interpreted similarly, such as “between” and “directly between”, “attached” and “directly attached”, “adjacent” and “directly adjacent”, etc.

It should be further noted that the terms “front”, “back”, “left”, “right”, “upper”, and “lower” are used in the following description to refer to directions in the drawings. The terms “inner” and “outer” are used to refer to directions towards and away from the geometric center of a particular component respectively. It will be understood that the terms so used are used herein to describe the relationship of one element, layer or region relative to another element, layer or region as illustrated in the figures. These terms should also encompass other orientations of the device in addition to the orientation depicted in the drawings.

Other aspects of the invention will be apparent to those skilled in the art in view of the disclosure herein.

In order to more clearly illustrate the embodiments of the present invention or the technical solutions in the prior art, specific embodiments of the present invention will be described below with reference to the accompanying drawings. It is obvious that the drawings in the following description are only some embodiments of the present invention. It is obvious for a person of ordinary skill in the art to obtain other drawings and other embodiments according to these drawings without involving any inventive effort.

It should be noted that the figures provided in the following examples merely illustrate the basic idea of the present application in a schematic way. Thus, only the components related to the present application are shown in the drawings instead of being drawn according to the number, shape and size of the components in an actual implementation. In an actual implementation, the type, number and proportion of the components may be changed at will, and the layout of the components may be more complicated. For example, the thicknesses of elements in the drawings may be exaggerated for clarity.

In the present invention, the inventors have conducted extensive and intensive experiments, and found that the present invention, based on the demand reference of artificial intelligence-assisted drug design, constructs a new process method to perform end-to-end SMILES sequence cleaning, deduplication, and conversion to mathematical figure standardization of small molecule compounds, and provides a more accurate and efficient data preprocessing method for a downstream artificial intelligence model. In order to achieve the above object, a first aspect of the invention provides a data preprocessing method for cleaning a small molecule compound, the data preprocessing method including: an S1 text preprocessing step including: preprocessing an original SMILES text of a small molecule compound into a standardized SMILES text of the small molecule compound according to predetermined text processing rules; and an S2 chemical graph formatting step including: splitting in a format each text element of the standardized SMILES text of the small molecule compound of S1 according to the predetermined text processing rules to obtain a digitized graph structure of chemical information of the small molecule compound.

In a preferred embodiment of the present invention, the method further includes a step S3, wherein the digitized graph structure of the chemical information of the small molecule compound of S2 is used for the construction of an artificial intelligence model. By way of example and not limitation, the final presentation is in Python list format and may be saved in Python pickle format for downstream deep learning training.

In a preferred embodiment of the present invention, when the original SMILES text of the small molecule compound is preprocessed into the standardized SMILES text of the small molecule compound in the S1 text preprocessing step, the predetermined text processing rules includes: step S1-1, optional structural normalization, wherein the data of the small molecule compound is processed into the original SMILES text; step S1-2, if the original SMILES text includes heavy metal components and organic compound components, removing the heavy metal components from and retaining the organic compound components in the original SMILES text; step S1-3, if the original SMILES text includes multimer components, removing the multimer components from and retaining a longest component in the original SMILES text; step S1-4, if the original SMILES text includes a charge, adding or subtracting a hydrogen atom in the original SMILES text to remove the charge; step S1-5, removing special SMILES text information; and step S1-6, exporting normalized sequences to obtain the normalized SMILES text for the small molecule compound.

More specifically, each section of the S1 step will be described below with reference to the accompanying drawings. The following description is given by way of illustration and not by way of limitation, and it is within the scope of the present invention for those skilled in the art to perform any combination of the following steps. Step S1-1, optional structural normalization, wherein the data of the small molecule compound is processed into the original SMILES text.

In a specific embodiment, raw data for the small molecule compound is entered, followed by a chemical structure normalization process, and finally processed into the original SMILES text (generally in text format). Specifically, when the chemical structure standardization is performed, text collating is performed using the predetermined text processing rules (Section S1-1). Specifically, the predetermined text processing rules (Section S1-1) include, but are not limited to the following. The text of the original data is modified to S1-1-1 standard text by the number rule. The regularization method is used to split all SMILES main components and recombine an SMILES text into an S1-1-2 standard text.

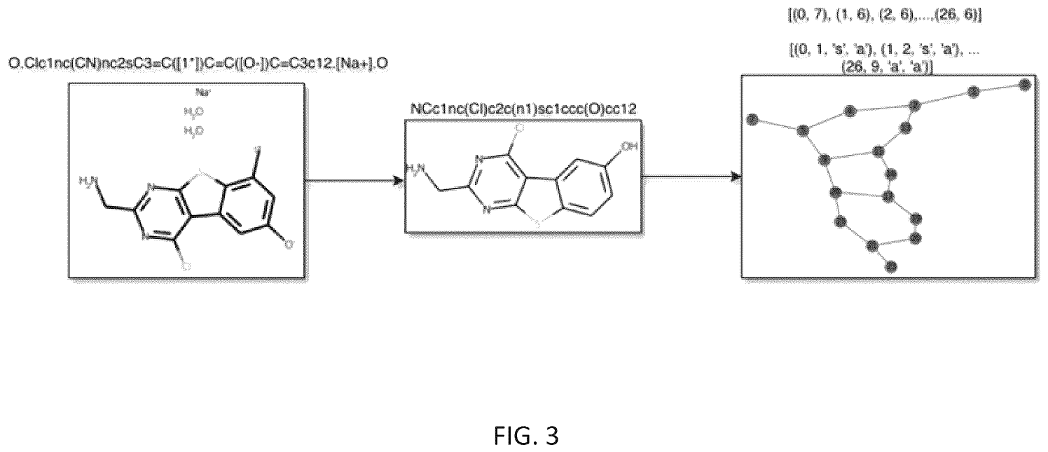

The process of recombination will use text rules to split the SMILES sequence components and then calculate the longest chain. The S1-1-3 standard text of the SMILES sequence is recombined by the longest chain. By way of example and not limitation, the S1-1-3 standard text is, for example, the SMILES sequence as shown in FIG. 3.

Step S1-2, if the original SMILES text includes heavy metal components and organic compound components, removing the heavy metal components from and retaining the organic compound components in the original SMILES text. Specifically, the S1-2 step is used to partially remove heavy metals from the SMILES text. More specifically, the section operates with text processing rules (Section S1-2). Herein, the heavy metal to be removed is defined as an atom without a covalent bond. By way of illustration and not limitation, the SMILES representation of a portion of the heavy metal atoms is SMILES text elements of atoms such as “[Li]”, “[Ca]”, “[Na+]”.

Step S1-3, if the original SMILES text includes multimer components, removing the multimer components from and retaining a longest component in the original SMILES text; Specifically, the purpose of the S1-3 step is to remove the multimer of SMILES text, with the longest sequence retained. More specifically, in the text, it will be split according to the separator “.”.

Step S1-4, if the original SMILES text includes a charge, adding or subtracting a hydrogen atom in the original SMILES text to remove the charge. Specifically, the purpose of step S1-4 is to zero out the charge components in the SMILES text. More specifically, this process can be understood as a text processing rule (Section S1-4). More specifically, the specific components in the covalent bond are modified. For example, “[O—]” is modified to “O”.

Step S1-5, removing special SMILES text information. The purpose of this step is to remove special marks or special atoms from the SMILES text. More specifically, this process can be understood as a text processing rule (Section S1-5). By way of example and not limitation, the modified text includes such as: “[1*]”, “*”, “[2H]”.

Step S1-6, exporting normalized sequences to obtain the normalized SMILES text for the small molecule compound. In a preferred embodiment of the present invention, when each text element of the standardized SMILES text of the small molecule compound of S1 is split in a format in the S2 chemical graph formatting step, the predetermined text processing rules include:
step S2-1, splitting the standardized SMILES text of the small molecule compound of S1 into text elements of each core to obtain text elements of the small molecule compound;
step S2-2, performing text processing and identification on the properties of the text elements of the small molecule compound of step S2-1, and identifying and completing simplified chemical information to obtain a chemical information graph of the small molecule compound;
step S2-3, according to the chemical information graph of the small molecule compound in step S2-2, establishing a coordinate system with an atomic element as a node, and constructing a digital coordinate system of the chemical information graph of the small molecule compound;
step S2-4, according to the digital coordinate system of the chemical information graph of the small molecule compound in step S2-3, adding element attributes of nodes and edges to obtain a digitized graph structure of the chemical information of the small molecule compound.
   More specifically, the step S2 is described below with reference to the drawings. The following description is given by way of illustration and not by way of limitation, and it is within the scope of the present invention for those skilled in the art to perform any combination of the following steps.

Step S2-1, splitting the standardized SMILES text of the small molecule compound of S1 into text elements of each core to obtain text elements of the small molecule compound. The purpose of the S2-1 step is to split the normalized SMILES sequence to each key text element (tokenization). Specifically, the text element includes a chemical bond label, an atom label, a chiral label, an organic compound ring label, or a combination thereof.

Step S2-2, performing text processing and identification on the properties of the text elements of the small molecule compound of step S2-1, and identifying and completing simplified chemical information to obtain a chemical information graph of the small molecule compound. The purpose of S2-2 is to complement the missing elements by a text processing rule algorithm. SMILES typically hides part of the information and this step will restore the hidden information to the default information. By way of illustration and not limitation, the complement of the ‘-’ element serves as a labeling element for a single bond compound covalent bond.

Step S2-3, according to the chemical information graph of the small molecule compound in step S2-2, establishing a coordinate system with an atomic element as a node, and constructing a digital coordinate system of the chemical information graph of the small molecule compound. The purpose of step S2-3 is to respectively mark nodes and edges with coordinates according to the order of element splitting. By way of example and not limitation, node elements are atoms, and edge elements are bonds. The coordinates of 0, . . . , N are marked sequentially by the input normalized SMILES sequence.

Step S2-4, according to the digital coordinate system of the chemical information graph of the small molecule compound in step S2-3, adding element attributes of nodes and edges to obtain a digitized graph structure of the chemical information of the small molecule compound. The purpose of step S2-4 is to construct a graph by integrating the information of nodes and edges as an initial mathematical graph via the coordinate system of step S2-3. By way of example and not limitation, the construction of the graph will take the coordinates of each node as a node list data structure. Matching of left and right nodes is performed by compound bond information (−, =, #, : and other elements) complemented by step 2 to create edges of a mathematical graph.

Alternatively, the nodes or edges may be specially marked by other marked elements as attributes in a mathematical graph, respectively. By way of example and not limitation, specific marks include, but are not limited to: attributes such as chiral atom marks (@, @@, /, \), atom numbers (inquired by rules), single-double triple bonds (see information in step 4), aromaticity (identified by rules), and whether within the compound ring (numeral recognition by regular expressions), etc.

In a preferred embodiment of the present invention, it further includes the step of S2-5, complementing the hydrogen atom information of the digitized graph structure of the chemical information, if necessary. By way of example and not limitation, the mathematical graph may be selectively added with hydrogen atoms. The completion of complementing method is based on rules of atomic attributes, and the relevant attribute information is complemented.

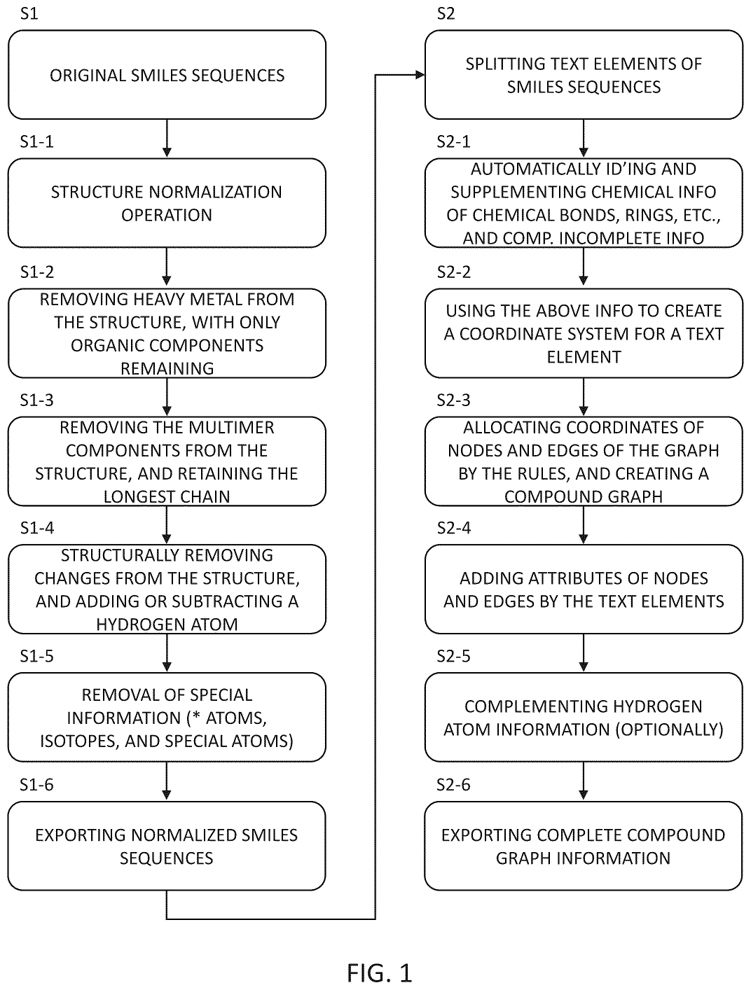

In a specific embodiment, it further includes step S2-6, completely exporting a digitized graph structure of chemical information of the small molecule compound. By way of example and not limitation, the chemical structure diagram shown in FIG. 3 is output. In particular, referring to FIG. 1, a preferred embodiment of the present invention is shown.

The idea of this preferred embodiment is as follows. This method is divided into two parts: text preprocessing, and text to mathematical graphs.

The text preprocessing includes:
1. structure standardization;
2. removing heavy metal components and retaining organic compound components in the structure text;
3. removing the multimer components from the structure text and retaining the longest component;
4. adding or subtracting a hydrogen atom in the structure text to remove the charge;
5. removing special SMILES text information;
6. exporting normalized sequences

The text to graph includes that:
1. the SMILES sequences are split to core elements;
2. by text processing, it identifies the nature of text elements and identifies and complements simplified chemical information;
3. a coordinate system with atomic elements as nodes is created, and a mathematical graph is constructed;
4. element attributes of nodes and edges are added;
5. hydrogen atom information is complemented

A complete compound graph is exported. More specifically, the S1 process is an upper part process, and the data output by this process can be saved or converted. The following is an explanation in detail.
1. Original SMILES data. The data format is text. The SMILES sequence is a textual representation of a small molecule compound, as in the case shown in FIG. 3.
2. Chemical structure standardization. Text collating is performed using the text processing rules. The original text is modified to the standard text in the method by the number rule. At the same time, the regularization method is used to split all SMILES main components and recombine an SMILES text into a standard text. The process of recombination will use text rules to split the SMILES sequence components and then calculate the longest chain. The SMILES sequence text is recombined by the longest chain.
3. The multimers of SMILES text are removed, with the longest sequence retained. The text will be split according to the separator “.”
4. Heavy metals are removed from the SMILES text. This section operates with text processing rules. Heavy metals are defined as atoms with no covalent bonds present. In the examples, the SMILES representation of a portion of the heavy metal atoms is SMILES text elements of atoms such as “[Li]”, “[Ca]”, “[Na+]”.
5. The charge components in the SMILES text are zeroed out. The method is performed using the text processing rules. The specific components in the covalent bond are modified by rules. For example, “[O—]” is modified to “O”.
6. Special marks and special atoms in the SMILES text are removed, and the text processing rules are also used by the method. The modified text includes such as: “[1*]”, “*”, “[2H]”.
7. The normalized SMILES sequences are exported.

The S2 process in FIG. 1 is the next half of the process, where the input is the SMILES sequence and the output is the mathematical graph formatting variables.
1. The normalized SMILES sequence is split to each tokenization. The element includes a chemical bond label, an atom label, a chiral label, and an organic compound ring label. The missing elements are complemented by a text processing rule algorithm. SMILES typically hides part of the information and this step will restore the hidden information to the default information. For example, the complement of the ‘-’ element serves as a labeling element for a single bond compound covalent bond.
The nodes and edges are respectively marked with coordinates by splitting the order of the elements. In an example, the node elements are atoms and the edge elements are bonds. The coordinates of 0, . . . , N are marked sequentially by the input normalized SMILES sequence.
4. The coordinate system of step 3 integrates the information of nodes and edges as an initial mathematical graph to construct a graph.
The construction of the graph will take the coordinates of each node as a node list data structure. Matching of left and right nodes is performed by compound bond information (−, =, #, : and other elements) complemented by step 2 to create edges of a mathematical graph.
5. The nodes or edges are specially marked by other marked elements as attributes in the mathematical graph. In examples, specific marks include, but are not limited to: attributes such as chiral atom marks (@, @@, /, \), atom numbers (inquired by rules), single-double triple bonds (see information in step 4), aromaticity (identified by rules), and whether within the compound ring (numeral recognition by regular expressions), etc.
6. (Optionally) hydrogen atom information is complemented. In the examples, the mathematical graph may be selectively added with hydrogen atoms. The completion of complementing method is based on rules of atomic attributes, and the relevant attribute information is complemented.
7. The exported chemical structure diagram is the final illustration of FIG. 3.

In a preferred embodiment of the present invention, the method further includes a step S3, wherein the digitized graph structure of the chemical information of the small molecule compound of S2 is used for the construction of an artificial intelligence model.

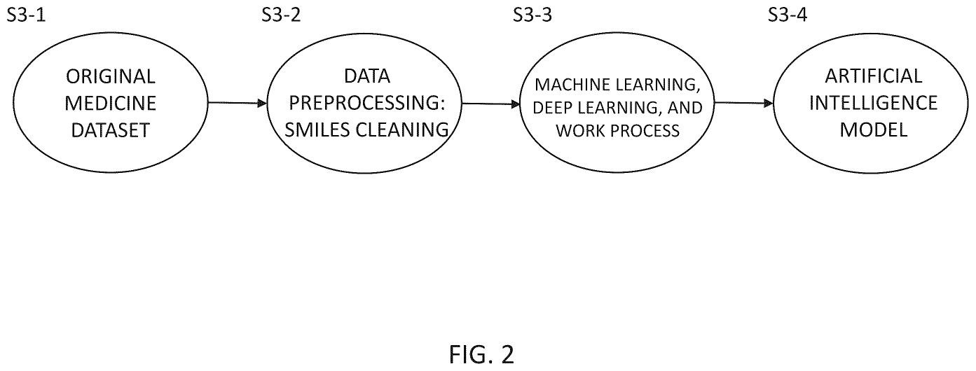

Referring to FIG. 2, the workflow of step S3 is shown. Step S3 includes:
S3-1 obtaining the original medicine dataset;
S3-2 data preprocessing (SMILES cleaning);
S3-3 work process of machine learning and deep learning;
S3-4: artificial intelligence model.

The S3 step is described by examples below.

### Example 1

1. An SMILES sequence dataset is input.
2. Each sequence is respectively subjected to the S1 process shown in FIG. 1. It decides whether to normalize part of optional steps based on the parameters.
3. Parallel computing is arranged by machine resource allocation to improve computing efficiency.
4. Datasets for cleaning SMILES are output and stored for other purposes. The storage method includes SQL database or csv, excel and other tabular formats.

### Example 2

1. An SMILES sequence dataset is input.
2. Each sequence is separately subjected to the S1 procedure shown in FIG. 1.
3. Parallel computing is arranged by machine resource allocation to improve computing efficiency.
4. Datasets for cleaning SMILES are output.
5. Each cleaned SMILES sequence is respectively subjected to the S2 process shown in FIG. 1.
6. Parallel computing is arranged by machine resource allocation to improve computing efficiency.
7. All compound graph data variables are exported. The entire dataset is presented in a python list format, with each mathematical graph having a node list variable and an edge list variable as shown in FIG. 3.
8. As shown in the last two steps of FIG. 2, the data is saved for machine learning and deep learning training in a python pickle format.

Taking FIG. 3 as an example, the overall process in some examples is as follows:
1. The data in SMILES format originally from a source is input.
2. The S1 process shown in FIG. 1 is performed.
3. The S2 process shown in FIG. 1 is performed to output mathematical graph data variables that can be used for modeling.

Specifically, the final presentation results are in Python list format, which can be saved as Python pickle format for downstream deep learning training.

In summary, the method implements global dataset cleaning, de-duplication, and normalization as compared to the original SMILES sequence text. The samples with conflict and different original data are uniformly standardized for downstream analysis. Compared with the traditional ETL data processing method, this method realizes the transformation from original data to data that can be used for training, and standardizes the workflow from original data to the training dataset to the data model training.

A second aspect of the present invention provides a data preprocessing system for cleaning a small molecule compound for use in the data preprocessing method of the present invention, including:
an S1 text preprocessing unit configured to include preprocessing original SMILES data of the small molecule compound into a standardized SMILES text of the small molecule compound according to predetermined text processing rules; and
an S2 chemical graph formatting unit configured to include: splitting in a format each text element of the standardized SMILES text of the small molecule compound of S1 according to the predetermined text processing rules to obtain a digitized graph structure of chemical information of the small molecule compound.

In a preferred embodiment of the present invention, it further includes an S3 unit configured such that a digitized graph structure of the chemical information of the small molecule compound of S2 is used in the construction of an artificial intelligence model.

In a preferred embodiment of the present invention, when the original SMILES text of the small molecule compound is preprocessed into the standardized SMILES text of the small molecule compound in the S1 text preprocessing unit, the predetermined text processing rule includes:
an S1-1 unit configured for optional structural normalization, wherein the data of the small molecule compound is processed into the original SMILES text;
an S1-2 unit configured for, if the original SMILES text includes heavy metal components and organic compound components, removing the heavy metal components from and retaining the organic compound components in the original SMILES text;
an S1-3 unit configured for, if the original SMILES text includes multimer components, removing the multimer components from and retaining a longest component in the original SMILES text;
an S1-4 unit configured for, if the original SMILES text includes a charge, adding or subtracting a hydrogen atom in the original SMILES text to remove the charge;
an S1-5 unit configured for removing special SMILES text information; and
an S1-6 unit configured for exporting normalized sequences to obtain the normalized SMILES text for the small molecule compound.

In a preferred embodiment of the present invention, when each text element of the standardized SMILES text of the small molecule compound of S1 is split in a format in the S2 chemical graph formatting unit, the predetermined text processing rules include:
an S2-1 unit configured for splitting the standardized SMILES text of the small molecule compound of S1 into text elements of each core to obtain text elements of the small molecule compound;
an S2-2 unit configured for performing text processing and identification on the properties of the text elements of the small molecule compound of the S2-1 unit, and identifying and completing simplified chemical information to obtain a chemical information graph of the small molecule compound;
an S2-3 unit configured for, according to the chemical information graph of the small molecule compound in the S2-2 unit, establishing a coordinate system with an atomic element as a node, and constructing a digital coordinate system of the chemical information graph of the small molecule compound;
an S2-4 unit configured for, according to the digital coordinate system of the chemical information graph of the small molecule compound in the S2-3 unit, adding element attributes of nodes and edges to obtain a digitized graph structure of the chemical information of the small molecule compound.

In a preferred embodiment of the invention, an S2-5 unit configured for, if necessary, complementing the hydrogen atom information of the digitized graph structure of the chemical information, if necessary. In a specific embodiment, it further includes a unit S2-6, completely exporting a digitized graph structure of chemical information of the small molecule compound. A third aspect of the invention provides an electronic device including a memory and a processor, wherein the memory is configured to store one or more computer instructions which, when executed by the processor, implement the data preprocessing method for cleaning a small molecule compound of the present invention.

Based on the present application, one skilled in the art will appreciate that one aspect described herein can be implemented independently of any other aspects, and that two or more of these aspects can be combined in various ways. For example, an apparatus may be implemented and/or a method practiced using any number and aspects set forth herein. Additionally, such an apparatus can be implemented and/or such a method can be practiced using other structures and/or functionality in addition to one or more of the aspects set forth herein.

Those skilled in the art will appreciate that, in addition to the system and its various devices, modules, and units provided by the present invention being implemented as purely computer readable program code, the system and its various devices, modules, and units provided by the present invention may well be implemented in the form of logic gates, switches, application specific integrated circuits, programmable logic controllers, embedded micro-controllers, etc. by logically programming the method steps. Therefore, the system and various devices, modules and units thereof provided by the present invention can be considered as a hardware component, and the devices, modules and units for realizing various functions included therein can also be considered as structures within the hardware component. The devices, modules and units for performing a function can also be considered structures within both a software module and a hardware component for performing a method.

It should be noted that the above embodiments can be freely combined as required. The above mentioned are only preferred embodiments of the invention. It will be appreciated by those skilled in the art that some modifications and adaptations may be made without departing from the principle of the invention, and such modifications and alterations are intended to be included within the scope of the invention.

All documents mentioned herein are incorporated herein by reference as if each document were individually incorporated by reference. Furthermore, it will be appreciated that those skilled in the art, upon reading the foregoing description of the invention, may make various changes and modifications to the invention, and all such equivalents are intended to fall within the scope of the appended claims.

1. A data preprocessing method for cleaning a small molecule compound, characterized in that the data preprocessing method comprising:
a step S1, text preprocessing step, including: preprocessing an original SMILES text of the small molecule compound into a standardized SMILES text of the small molecule compound according to predetermined text processing rules; and
a step S2, chemical graph formatting step, including: splitting in a format each text element of the standardized SMILES text of the small molecule compound of S1 according to the predetermined text processing rules to obtain a digitized graph structure of chemical information of the small molecule compound.

2. The data preprocessing method for cleaning a small molecule compound of claim 1, further comprising:
a step S3, wherein the digitized graph structure of the chemical information of the small molecule compound of step S2 is used for the construction of an artificial intelligence model.

3. The data preprocessing method for cleaning a small molecule compound of claim 1, wherein when the original SMILES text of the small molecule compound is preprocessed into the standardized SMILES text of the small molecule compound in the S1 text preprocessing step, the predetermined text processing rules comprises:
step S1-1, optional structural normalization, wherein the data of the small molecule compound is processed into the original SMILES text;
step S1-2, respondent to the original SMILES text comprises heavy metal components and organic compound components, removing the heavy metal components from and retaining the organic compound components in the original SMILES text;
step S1-3, respondent to the original SMILES text comprises multimer components, removing the multimer components from and retaining a longest component in the original SMILES text;
step S1-4, respondent to the original SMILES text comprises a charge, adding or subtracting a hydrogen atom in the original SMILES text to remove the charge;
step S1-5, removing special SMILES text information; and
step S1-6, exporting normalized sequences to obtain the normalized SMILES text for the small molecule compound.

4. The data preprocessing method for cleaning a small molecule compound of claim 1, further comprising:
respondent to each text element of the standardized SMILES text of the small molecule compound of S1 is split in a format in the S2 chemical graph formatting step, the predetermined text processing rules comprises:
step S2-1, splitting the standardized SMILES text of the small molecule compound of S1 into text elements of each core to obtain text elements of the small molecule compound;
step S2-2, performing text processing and identification on the properties of the text elements of the small molecule compound of step S2-1, and identifying and completing simplified chemical information to obtain a chemical information graph of the small molecule compound;
step S2-3, according to the chemical information graph of the small molecule compound in step S2-2, establishing a coordinate system with an atomic element as a node, and constructing a digital coordinate system of the chemical information graph of the small molecule compound; and
step S2-4, according to the digital coordinate system of the chemical information graph of the small molecule compound in step S2-3, and adding element attributes of nodes and edges to obtain a digitized graph structure of the chemical information of the small molecule compound.

5. The data preprocessing method for cleaning a small molecule compound of claim 4, further comprising:
step S2-5, complementing the hydrogen atom information of the digitized graph structure of the chemical information, if necessary.

6. A data preprocessing system for cleaning a small molecule compound adapted for a data preprocessing method for cleaning a small molecule compound, characterized in that the data preprocessing method comprising:
a step S1, text preprocessing step, including: preprocessing an original SMILES text of the small molecule compound into a standardized SMILES text of the small molecule compound according to predetermined text processing rules; and
a step S2, chemical graph formatting step, including: splitting in a format each text element of the standardized SMILES text of the small molecule compound of S1 according to the predetermined text processing rules to obtain a digitized graph structure of chemical information of the small molecule compound;
wherein the system comprises:
an S1 text preprocessing unit configured to include preprocessing original SMILES data of the small molecule compound into a standardized SMILES text of the small molecule compound according to predetermined text processing rules; and
an S2 chemical graph formatting unit configured to include: splitting in a format each text element of the standardized SMILES text of the small molecule compound of S1 according to the predetermined text processing rules to obtain a digitized graph structure of chemical information of the small molecule compound.

7. The data preprocessing system for cleaning a small molecule compound of claim 6, further comprising an S3 unit configured such that a digitized graph structure of the chemical information of the small molecule compound of S2 is used in the construction of an artificial intelligence model.

8. The data preprocessing system for cleaning a small molecule compound of claim 6, when the original SMILES text of the small molecule compound is preprocessed into the standardized SMILES text of the small molecule compound in the S1 text preprocessing unit, the predetermined text processing rule comprises:
an S1-1 unit configured for optional structural normalization, wherein the data of the small molecule compound is processed into the original SMILES text;
an S1-2 unit configured for, if the original SMILES text comprises heavy metal components and organic compound components, removing the heavy metal components from and retaining the organic compound components in the original SMILES text;
an S1-3 unit configured for, if the original SMILES text comprises multimer components, removing the multimer components from and retaining a longest component in the original SMILES text;
an S1-4 unit configured for, if the original SMILES text comprises a charge, adding or subtracting a hydrogen atom in the original SMILES text to remove the charge;
an S1-5 unit configured for removing special SMILES text information; and
an S1-6 unit configured for exporting normalized sequences to obtain the normalized SMILES text for the small molecule compound.

9. The data preprocessing system for cleaning a small molecule compound of claim 6, when each text element of the standardized SMILES text of the small molecule compound of S1 is split in a format in the S2 chemical graph formatting unit, the predetermined text processing rules comprises:
an S2-1 unit configured for splitting the standardized SMILES text of the small molecule compound of S1 into text elements of each core to obtain text elements of the small molecule compound;
an S2-2 unit configured for performing text processing and identification on the properties of the text elements of the small molecule compound of the S2-1 unit, and identifying and completing simplified chemical information to obtain a chemical information graph of the small molecule compound;
an S2-3 unit configured for, according to the chemical information graph of the small molecule compound in the S2-2 unit, establishing a coordinate system with an atomic element as a node, and constructing a digital coordinate system of the chemical information graph of the small molecule compound; and
an S2-4 unit configured for, according to the digital coordinate system of the chemical information graph of the small molecule compound in the S2-3 unit, adding element attributes of nodes and edges to obtain a digitized graph structure of the chemical information of the small molecule compound.

10. The data preprocessing system for cleaning a small molecule compound of claim 9, further comprising:
an S2-5 unit configured for, if necessary, complementing the hydrogen atom information of the digitized graph structure of the chemical information.

11. An electronic device comprising:
a memory; and
a processor, wherein the memory is configured to store one or more computer instructions which, when executed by the processor, implements a data preprocessing method for cleaning a small molecule compound, wherein the data preprocessing method comprises:
a step S1, text preprocessing step, including: preprocessing an original SMILES text of the small molecule compound into a standardized SMILES text of the small molecule compound according to predetermined text processing rules; and
a step S2, chemical graph formatting step, including: splitting in a format each text element of the standardized SMILES text of the small molecule compound of S1 according to the predetermined text processing rules to obtain a digitized graph structure of chemical information of the small molecule compound.
